(19) United States
(12) Patent Application Publication
(10) Pub. No.: US 2010/0297704 A1
(43) Pub. Date: Nov. 25, 2010

(54) PROCESS FOR BIOMASS CONVERSION

(76) Inventor: Rongxiu LI, Shanghai (CN)

Correspondence Address: LAHIVE & COCKFIELD, LLP;FLOOR 30, SUITE 3000, ONE POST OFFICE SQUARE, BOSTON, MA, 02109, US

(21) Appl. No.: 12/707,994

(22) Filed: Feb. 18, 2010

Related U.S. Application Data
(60) Provisional application No. 61/153,517, filed on Feb. 18, 2009.

(51) Int. Cl. C13K 1/02; C13F 3/00; C12P 19/00; C12P 1/00

(52) U.S. Cl. 435/72; 127/37; 127/30; 435/41

(57) ABSTRACT

The present invention relates to a clean process of preparing high grade biomass products, and their use in the production of health care products, bio-energy products, biochemicals, bio-originated chemicals and biodegradable plastics.

### RELATED APPLICATIONS

This application claims priority to U.S. Provisional Application Ser. No. 61/153,517, entitled “Process For Biomass Conversion”, filed on Feb. 18, 2009. The entire content of this application is incorporated herein by reference.

### BACKGROUND

Lignocellulosic biomass is one of the most abundant and renewable forms of biomass that is built up through solar-powered photosynthesis. A 2005 joint report from the U.S. Departments of Energy and Agriculture found that more than 1 billion tons of biomass could be available in the U.S. to produce biofuels and bioproducts, which is enough to meet 30 percent of U.S. demand for transportation fuels and 25 percent of demand for chemicals [Biomass as feedstock for a bioenergy and bioproducts industry: the technical feasibility of a billion-ton annual supply, 2005, DOE & USDA, available electronically at: http://www.osti.gov/bridge]. In China, there is also about one billion tons of lignocellulosic biomass available annually. Lignocellulosic biomass comes in many different forms, which can be grouped into four main categories: (1) forest products and wood residues; (2) agricultural residues (including corn stover, wheat and rice straw and sugarcane bagasse); (3) dedicated energy crops (which are mostly composed of fast growing, tall, and woody grasses); and/or (4) municipal garden waste and paper waste.

A typical composition of lignocellulosic biomass includes around 20-40% cellulose, 15-40% hemicellulose, 20-30% lignin, and less than 8% extractives of proteins and natural compounds depending on the type and source of the raw biomass. However, most lignocellulosic biomass is treated as waste and discarded for degradation. For example, in China, wheat straw, rice straw, and corn stover are usually burned or left to rot in open fields, leaking waste water that pollutes rivers.

Accordingly, there remains a need for new methods to convert the cellulose, hemicellulose, lignin, or other components in lignocellulosic biomass into bioenergy and/or biochemicals, as well as methods to reduce lignocellulosic waste.

### SUMMARY OF THE INVENTION

The present invention addresses the need for overcoming the technology bottleneck in the preparation of clean, green and/or highly valuable biochemical, chemical, and health-care products from lignocellulosic biomass. Specifically, the present invention relates to a clean process of preparing high grade biomass products, and their use in the production of health care products, bio-energy products, biochemicals, bio-originated chemicals and biodegradable plastics. The process comprises the use of acids to break biomass recalcitrance by hydrolysis of hemicelluloses and pectin, forming soluble xylose polymer and xylose. After extraction, the remaining solid residue is conditioned to become very fragile and is easily grinded into fine particles, so that the cellulose component in the grinded residue can be hydrolyzed by a cellulase enzyme at a rate enhanced by many folds to obtain high grade lignin and glucose. The high grade lignin can be further thermolyzed into high grade mixtures of BTX chemicals, which are valuable for individual BTX chemical production. These high grade forms of xylose, glucose, lignin and BTX chemicals are good starting materials for other products of high value, such as bio-fuel, biochemicals and bio-originated chemicals.

Thus, in one aspect, the invention provides a process for converting biomass into soluble and insoluble fractions, said process comprising:

(i) incubating the biomass with an aqueous acidic liquor;

(ii) separating the acidic liquor from the resulting solid residue of the biomass,

wherein the acidic liquor contains the soluble fraction and the solid residue contains the insoluble fraction; and

(iii) collecting the products of the biomass conversion.

In one embodiment of the process above, the incubation with the acidic liquor produces a soluble product, or a combination of products, separated from the biomass and dissolved in the acidic liquor. In another embodiment of the process above, the process further comprises separating the soluble product(s) from the acidic liquor. In still another embodiment, the acidic liquor is recovered after step (ii) for reuse.

In another aspect, the invention provides a process for converting biomass into soluble and insoluble products, said process comprising:

(i) incubating the biomass with an aqueous acidic liquor so that a soluble product, or a combination of soluble products, are separated from the biomass and dissolved in the acidic liquor;

(ii) separating the acidic liquor containing the soluble product(s) from the resulting solid residue of the biomass;

(iii) recovering the soluble products from the acidic liquor;

(iv) recovering the acidic liquor from the separated liquid and the solid residue for reuse;

(v) grinding the insoluble solid residue recovered from step (ii) and further hydrolyzing the insoluble solid residue with cellullase enzyme; and

(vi) recovering the resulting soluble products and the insoluble lignin products from steps (ii)-(iii) and the resulting solid residue.

In one preferred embodiment of the processes above, the biomass comprises a lignocellulosic biomass feedstock.

In another aspect, the invention provides a process for converting lignocellulosic biomass feedstock into lignocellulosic products, the process comprising:

(i) incubating the lignocellulosic biomass feedstock with an acidic liquor comprising an acid or a combination of acids so that a soluble product, or a combination of products, are separated from the biomass and dissolved in the acid(s);

(ii) separating the resulting liquid from the resulting solid residue, wherein the liquid contains the soluble lignocellulosic products and the solid residue contains the insoluble lignocellulosic fraction;

(iii) recovering the soluble lignocellulosic products from the separated liquid;

(iv) recovering the acidic liquor from the separated liquid for reuse;

(v) grinding the insoluble lignocellulosic solid fraction recovered in step (iii) and further hydrolyzing the insoluble lignocellulosic solid residue with cellullase enzyme; and

(vi) recovering the resulting soluble products and the insoluble lignin products as in steps (ii)-(iii) and the resulting solid residue.

In one embodiment of the process above, the lignocellulosic biomass feedstock comprises fragmentated feedstock. In another embodiment of the process above, the lignocellulosic biomass is capable of passing through about 8-64 mesh filters. In still another embodiment of the process above, the weight:volume ratio of said lignocellulosic biomass feedstock vs. acid is 1:2-1:20. In yet another embodiment of the process above, the lignocellulosic biomass feedstock in step (i) is incubated for 1-16 hours. In just another embodiment of the process above, the lignocellulosic biomass feedstock in step (i) is incubated with 0.5% trifluoroacetic acid (TFA) at 90° C. for 16 hours. In another embodiment of the process above, the lignocellulosic biomass feedstock in step (i) is incubated with 70% trifluoroacetic acid (TFA) at 90° C. for 5 hours. In another embodiment of the process above, the lignocellulosic biomass feedstock in step (i) is incubated with 0.1%-5% dilute acid at 50° C.-90° C. and in step (v) the insoluble lignocellulosic solid fraction is conditioned before grinding at temperature 120° C.-160° C. In a preferred embodiment, the insoluble lignocellulosic solid fraction conditioned in step (v) is heated for 3-6 hours. In another embodiment of the process above, the separated liquid in step (iii) contains soluble xylose polymer, xylose oligomer, or xylose monomer, or a combination thereof, in the concentration of at least 90%. In another embodiment of the process above, the hydrolysis in step (v) comprises an incubation with cellulase at 10° C.-90° C. In still another embodiment of the process above, the cellulase in step (v) is used for hydrolysis to produce soluble products comprising glucose, wherein the concentration of produced glucose is at least 90%. In yet another embodiment of the process above, the cellulase in step (v) is used for hydrolysis and the resulting solid residue comprises lignin, wherein the concentration of lignin in said solid residue is at least 90%.

In one embodiment of above processes, the biomass is selected from the group consisting of woody plants, gramineous plants, and herbage plants, or a combination thereof. In another embodiment of the processes, the biomass is converted to products selected from the group consisting of cellulose, hemicellulose, xylose polymer, xylose oligomer, xylose monomer, glucose, lignin, and other lignocellulosic products, or a combination thereof. In another embodiment of the processes, the converted products are further converted to bioenergy, biochemicals, or other bulk materials, or a combination thereof. In a preferred embodiment, the converted products are further converted to bioenergy, biochemicals, or other bulk materials, or a combination thereof.

In one embodiment of above processes, the acidic liquor in step (i) comprises a low-boiling point organic acid or an inorganic (mineral) acid, or a combination thereof. In a preferred embodiment, the organic acid in step (i) is selected from the group consisting of formic acid, acetic acid, 2-hydroxypropionic acid, propionic acid, acrylic acid, propylene-2-carboxylic acid, n-pentanoic acid, lactic acid, trifluoromethane sulfonic acid, methyl acrylic acid and trifluoroacetic acid (TFA), or a combination thereof. In a most preferred embodiment, the said organic acid comprises trifluoroacetic acid (TFA). In another embodiment of the processes, the inorganic acid in step (i) is selected from the group consisting of hydrochloric acid, sulfuric acid, phosphoric acid, and nitric acid, or a combination thereof.

In one embodiment of the above processes, the biomass is incubated with an aqueous acidic liquor comprising 0.1%-100% dilute acid. In a preferred embodiment, the biomass is incubated with an aqueous acidic liquor comprising 0.1%-5.0% dilute acid. In another embodiment of the above processes, the biomass is incubated at about 50° C.-160° C. In still another embodiment of the above processes, the soluble products converted from said biomass in step (vi) is glucose with a purity of at least 90%. In yet another embodiment of the above processes, the insoluble products converted from said biomass in step (vi) is lignin with a purity of at least 90%.

In one embodiment of the above processes, the recovered lignin products are further converted into lignin-related products. In one preferred embodiment, the recovered lignin products are heated with or without catalyst to 300° C.-500° C. In another preferred embodiment, the recovered lignin products are heated to 450° C. under vacuum for 12 hours to produce a resulting liquid comprising at least 5 compounds with a total concentration of at least 75%. In still another preferred embodiment, the recovered lignin products are mixed with Al₂O₃ and Fe₂O₃ as catalyst and heated to 400° C. under vacuum for 12 hours to produce a resulting liquid comprising at least 5 compounds with a total content of at least 79%. In still another embodiment of the process above, the lignin is thermolyzed into substituted coniferols, propylphenol, eugenol, syringols, aryl ethers, or alkylated methyl aryl ethers, or a combination thereof.

In another aspect, this invention is related to a composition comprising the fractions or products produced by the above processes.

In another aspect, this invention is related to a process for converting lignocellulosic biomass feedstock into bioenergy, biochemicals, or other bulk materials, the process comprising:

(i) preparing xylose products using one or more of the above processes;

(ii) culturing at least one species of microbes in a both containing the prepared xylose products and fermenting; and

(iii) collecting bioenergy, biochemicals, or other bulk materials, or a combination thereof, from the fermentation broth.

In one embodiment of the process above, the microbes comprise Pichia pastoris GS115. In another embodiment of the process above, the fermentation of the microbe culture is used for said conversion.

In another aspect, this invention is related to a process for converting lignocellulosic biomass feedstock into xylose oligomer, the process comprising:

(i) preparing soluble lignocellulosic products with the above processes; and

(ii) separating xylose oligomers from the soluble lignocellulosic products in step (i).

In one embodiment of the process above, the separation is through ethanol precipitation. In a preferred embodiment, the concentration of ethanol is 30%-90%.

In another embodiment of the process above, the average polymerization degree of xylose oligomers is 1.3-5.6.

In another aspect, this invention is related to a lignocellulosic composition of products produced by the above processes.

In another aspect, this invention is related to a lignocellulosic composition comprising at least 90% of xylose polymer, xylose oligomer, xylose monomer, or a combination thereof, produced by the above processes.

In another aspect, this invention is related to a lignocellulosic composition comprising at least 90% of glucose, produced by the above processes.

In another aspect, this invention is related to a lignocellulosic composition comprising at least 90% of lignin, produced by the above processes.

In another aspect, this invention is related to a lignocellulosic feedstock processing system comprising a set of devices capable of carrying out the above processes. In one embodiment, the system further comprises a feedstock handling device and a preconditioner capable of receiving said feedstock from said handling device, wherein said preconditioner is in communication with said set of devices.

### BRIEF DESCRIPTION OF THE DRAWINGS

FIG. 1 illustrates one embodiment of the process for conversion of biomass feedstock.

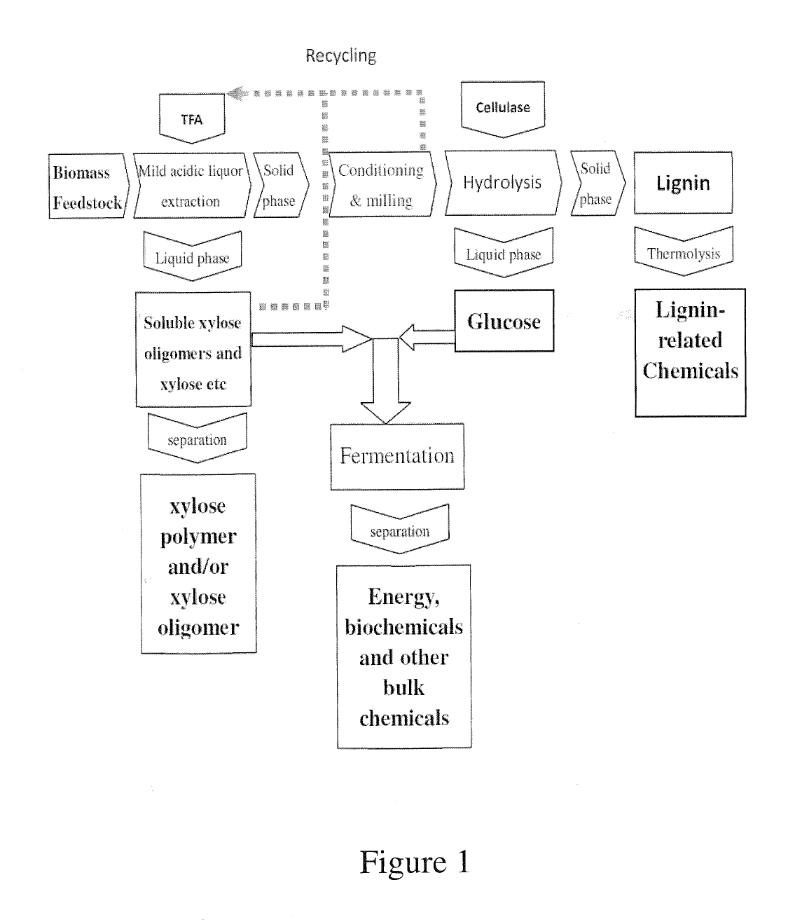

### DETAILED DESCRIPTION OF THE INVENTION

Cellulose, hemicelluloses and lignin, or their basic building monomers (glucose for cellulose; xylose for hemicelluloses; and substituted coniferols, propylphenol, eugenol, syringols, aryl ethers, alkylated methyl aryl ethers for lignin) exist naturally in the environment. If they could be prepared in high grade form, their value could be multiplied many times, as they are good starting materials for other products of high value, such as bio-fuel, biochemicals and bio-originated chemicals. One example is glucose fermentation to other bulk chemicals, including building blocks of 1,4-diacids (succinic, fumaric and malic), 2,5-furan dicarboxylic acid, 3-hydroxy propionic acid, aspartic acid, glucaric acid, glutamic acid, itaconic acid, levulinic acid, 3-hydroxybutyrolactone, glycerol, sorbitol, and xylitol/arabinitol, which can be subsequently converted to more than 100 high-value bio-based chemicals or materials including polylactic acid (PLA) and polyhydroxyalkanoates (PHAs) (Top Value Added Chemicals from Biomass Volume I—Results of Screening for Potential Candidates from Sugars and Synthesis Gas, T. Werpy and G. Petersen, Editors, available electronically at http://www.osti.gov/bridge). Xylose is a valuable raw material in the flavoring, functional food and fodder industry. It promotes the growth of human and animal intestinal twin-bacilli and improves human and animal microorganism immunity. It could be used to produce xylitol and is applied widely in food processing and medical industries. Xylose is widely used to manufacture xylitol by chemical or biochemical reduction. It is widely used for therapeutic purposes, such as tooth-decay prevention, because it cannot be degraded by cryogenic bacteria in oral cavity. Oligo-xylose is widely used as functional ingredient in health promoting food and food supplements.

High grade lignin can be turned into BTX chemicals (benzene, toluene, xylene), phenol, lignin monomer molecules (substituted coniferols: propylphenol, eugenol, syringols, aryl ethers, alkylated methyl aryl ethers), oxidized lignin monomers (syringaldehyde, vanillin, vanillic acid), new diacids and aromatic diacids, β-keto adipic acid, aliphatic acids, new polyesters, new polyols, aromatic polyols (cresols, catechols, resorsinols), cyclohexane and substituted cyclohexanes, and quinines. The potential value can be exemplified in various scenarios of lignin conversion predicted by U.S. Department of Energy (DOE). In one scenario, 1.5 million tons of lignin is converted to carbon fiber, while the remainder is converted to BTX chemicals and the byproducts of the process is converted to syngas alcohols. The revenue increase will be $35 billion, with an additional 8.6 billion gallons of ethanol produced (Top Value-Added Chemicals from Biomass Volume II—Results of Screening for Potential Candidates from Biorefinery Lignin, PNNL-16983). The DOE has also set a goal to replace thirty percent of the transportation fuel supply with biofuels by 2030, which equates to roughly 60 billion gallons of biofuel. Production of 60 billion gallons of ethanol, or other bio-derived fuel, will require the use of approximately 0.75 billion tons (1.5 billion pounds) of biomass. Because lignin constitutes up to 30% of the weight of biomass, it means that about 225 million tons (450 billion pounds) of lignin will be converted to biofuels and biochemicals.

Thus, the process of manufacturing high grade xylose, glucose and lignin from lignocellulosic biomass material holds remarkable potential to turn the most abundant renewable resources into high value bio-fuel, biochemicals, fine chemicals and other bulk chemicals. It would provide the dual advantage of a sustainable resource supply, not affecting food supplies, and all chemicals derived would have less environmental impact than petrochemicals. The “green” products such as ethanol, pharmaceutical intermediates, citric acid, and amino acids would grow from 5% to as high as ⅔ of the total global economy (Lucia, 2008, Lignocelluloses biomass: Replace petroleum, BioResources 3, 981-982).

The present invention addresses the need for new and clean methods of preparation of these bio-products, and others, from lignocellulosic biomass. Further, these bio-products can be usable for the production of clean and green energy and/or highly valuable chemical products. Specifically, the present invention relates to the use of acids to break biomass recalcitrance by hydrolysis of hemicelluloses and pectin, forming soluble xylose polymer and xylose. After extraction, the remaining solid residue is conditioned to become very fragile and is easily grinded into fine particles, so that the cellulose component in the grinded residue can be hydrolyzed by a cellulase enzyme at a rate enhanced by many folds to obtain high grade lignin and glucose. The high grade lignin can be further thermolyzed into BTX chemicals. As discussed above, the high grade lignin, glucose and xylose fractions are of high value by themselves and are good starting material to manufacture bioethanol, bio-diesel, citric acid, aspartic acid, amino acids, natural compounds, health care products, animal feedstuff, carbon fibre, and bulk chemicals, including building blocks for more than 300 high value chemicals.

Without being bound by theory, the current invention is based on the notion that the cellulose microfibrils and lignin in the cell wall are covalently cross-linked by hemicellulose to form the dense rind of grasses and bark of trees, making them resistant to mechanical breakage, microbial agents and enzyme penetration (C. Somerville et al., Science 306, 2206-11). As the weakest link in the lignocellulosic biomass structure, hemicelluloses can be broken up with relatively mild acid liquor. After the extraction of hemicelluloses, the remaining lignin component is conditioned to make it mechanically weak and fragile, and can then be easily grinded into fine fragments with minimum energy input. The increased total surface area after grinding contributes to the enhancement of the rate of cellulose hydrolysis with cellulase enzymes, and to the complete removal of cellulose from lignin. In the entire conversion process, low boiling point organic acids easily evaporate from the fractioned products (e.g., xylose, glucose and lignin), and the acids can be recovered for the next round of conversion process after condensing and recovery. Thus, there will be no discharge of waste acid, making the present invention a clean technology. In the case of dilute inorganic acid, the waste acid is cheap to recover economically, and can be neutralized forming harmless salt.

The resulting glucose, lignin, and poly-, oligo-, or monomer xylose in high purity grade are of higher value themselves, and are good starting materials for conversion into products described above, but not limited by the description.

Thus, the present invention provides a process for converting biomass into soluble and insoluble fractions, said process comprising:

(i) incubating the biomass with an aqueous acidic liquor;

(ii) separating the acidic liquor from the resulting solid residue of the biomass, wherein the acidic liquor contains the soluble fraction and the solid residue contains the insoluble fraction; and

(iii) collecting the products of the biomass conversion.

The present invention also provides a process for converting biomass into soluble and insoluble fractions, said process comprising:

(i) incubating the biomass with an aqueous acidic liquor;

(ii) separating the acidic liquor from the resulting solid residue of the biomass, wherein the acidic liquor contains the soluble fraction and the solid residue contains the insoluble fraction;

(iii) recovering the acidic liquor after step (ii) for reuse; and

(iv) collecting the products of the biomass conversion.

The present invention also provides a process for converting biomass into soluble and insoluble fractions, and preparation of xylose polymer and/or xylose oligomer, said process comprising:

(i) incubating the biomass with an aqueous acidic liquor;

(ii) separating the acidic liquor from the resulting solid residue of the biomass, wherein the acidic liquor contains the soluble fraction and the solid residue contains the insoluble fraction;

(iii) separating xylose polymer and/or xylose oligomer products from the acidic liquor containing the soluble fraction; and

(iv) collecting the products of the biomass conversion.

The present invention also provides a process for converting biomass into soluble and insoluble fractions, and preparation of xylose polymer and/or xylose oligomer, said process comprising:

(i) incubating the biomass with an aqueous acidic liquor;

(ii) separating the acidic liquor from the resulting solid residue of the biomass, wherein the acidic liquor containing the soluble fraction and the solid residue containing the insoluble fraction;

(iii) recovering the acidic liquor after step (ii) for reuse;

(iv) separating xylose polymer and/or xylose oligomer products from the acidic liquor containing the soluble fraction; and

(v) collecting the products of the biomass conversion.

The present invention also provides a process for converting biomass into soluble and insoluble products, said process comprising:

(i) incubating the biomass with an aqueous acidic liquor so that a soluble product, or a combination of products, are separated from the biomass and dissolved in the acidic liquor;

(ii) separating the acidic liquor containing the soluble products from the resulting solid residue of the biomass;

(iii) recovering the soluble products from the acidic liquor;

(iv) recovering the acidic liquor from the separated liquid and the solid residue for reuse;

(v) grinding the insoluble lignocellulosic solid fraction recovered in step (ii) and further hydrolyzing the cellulose within the insoluble residue with cellullase enzyme; and

(vi) recovering the resulting soluble products as in steps (ii)-(iii) and the resulting insoluble lignin products.

This invention also provides a process for converting lignocellulosic biomass feedstock into lignocellulosic products, the process comprising:

(i) incubating the lignocellulosic biomass feedstock with an acidic liquor comprising an acid or a combination of acids so that a soluble product, or a combination of products, are separated from the biomass and dissolved in the acid(s);

(ii) separating the resulting liquid from the resulting solid residue, wherein the liquid contains the soluble lignocellulosic products and the solid residue contains the insoluble lignocellulosic fraction;

(iii) recovering the soluble lignocellulosic products from the separated liquid;

(iv) recovering the acidic liquor from the separated liquid for reuse;

(v) conditioning and grinding the insoluble lignocellulosic solid fraction recovered in step (ii) and further hydrolyzing the cellulose within the insoluble residue with cellullase enzyme; and

(vi) recovering the resulting soluble products as in steps (ii)-(iii) and the resulting insoluble lignin products.

This invention also provides a process for converting lignocellulosic biomass feedstock into lignocellulosic products, and further converting the resulting lignocellulosic products to bioenergy, biochemicals and other bulk chemicals, the process comprising:

(i) incubating the biomass with an aqueous acidic liquor so that a soluble product, or a combination of products, are separated from the biomass and dissolved in the acidic liquor;

(ii) separating the acidic liquor containing the soluble products from the resulting solid residue of the biomass;

(iii) recovering the soluble products from the acidic liquor;

(iv) recovering the acidic liquor from the separated liquid and the solid residue for reuse;

(v) grinding the insoluble lignocellulosic solid fraction recovered in step (ii) and further hydrolyzing the insoluble solid residue with cellullase enzyme;

(vi) culturing at least one microbe with the recovered soluble products;

(vii) fermenting the composition of claim (vii); and

(viii) collecting fermentation products from the fermentation broth of step (vii).

This invention also provides a process for converting lignocellulosic biomass feedstock into lignocellulosic products, and further converting the resulting lignocellulosic products to bioenergy, biochemicals and other bulk chemicals, the process comprising:

(i) incubating the lignocellulosic biomass feedstock with an acidic liquor comprising an acid or a combination of acids so that a soluble product, or a combination of products, are separated from the biomass and dissolved in the acid(s);

(ii) separating the resulting liquid from the resulting solid residue, wherein the liquid contains the soluble lignocellulosic products and the solid residue contains the insoluble lignocellulosic fraction;

(iii) recovering the soluble lignocellulosic products from the separated liquid;

(iv) recovering the acidic liquor from the separated liquid for reuse;

(v) conditioning and grinding the insoluble lignocellulosic solid fraction recovered in step (ii) and further hydrolyzing the insoluble lignocellulosic solid residue with cellullase enzyme;

(vi) recovering the resulting soluble products and the insoluble lignin products as in steps (ii)-(iii) and the resulting solid residue;

(vii) culturing at least one species of microbes with the recovered soluble products and fermenting the resulting composition; and

(viii) collecting fermentation products from the fermentation broth of step (vii).

This invention also provides a process for converting biomass into water soluble and insoluble products, as well as lignin related liquid and solid products, said process comprising:

(i) incubating the biomass with an aqueous acidic liquor so that a soluble product, or a combination of products, are separated from the biomass and dissolved in the acidic liquor;

(ii) separating the acidic liquor containing the soluble products from the resulting solid residue of the biomass;

(iii) recovering the soluble lignocellulosic products from the acidic liquor;

(iv) recovering the acidic liquor from the separated liquid and the solid residue for reuse;

(v) grinding the insoluble lignocellulosic fraction recovered in step (ii) and further hydrolyzing the cellulose within the insoluble residue with cellullase enzyme;

(vi) recovering the resulting soluble products as in steps (ii)-(iii) and the resulting insoluble lignin products;

(vii) thermolyzing the insoluble lignin products recovered in step (vi); and

(viii) recovering the resulting lignin related liquid and solid products. This invention also provides a process for converting lignocellulosic biomass feedstock into lignocellulosic products, and lignin related liquid and solid products, the process comprising:

(i) incubating the lignocellulosic biomass feedstock with an acidic liquor comprising an acid or a combination of acids so that a soluble product, or a combination of products, are separated from the biomass and dissolved in the acid(s);

(ii) separating the resulting liquid from the resulting solid residue, wherein the liquid contains the soluble lignocellulosic products and the solid residue contains the insoluble lignocellulosic fraction;

(iii) recovering the soluble lignocellulosic products from the separated liquid;

(iv) recovering the acidic liquor from the separated liquid and the solid residue for reuse in step (i);

(v) conditioning and grinding the solid lignocellulosic fraction recovered in step (iii) and further hydrolyzing the fraction with cellullase enzyme;

(vi) recovering the resulting soluble products as in steps (ii)-(iii) and the resulting solid lignin products;

(vii) thermolyzing the solid lignin products recovered in step (vi); and

(viii) recovering the resulting lignin related liquid and solid products.

In a preferred embodiment, the biomass comprises a lignocellulosic biomass feedstock. In some preferred embodiments, the lignocellulosic biomass feedstock can, for example, include: (1) forest products and wood residues; (2) agricultural residues (including corn stover, wheat and rice straw and sugarcane bagasse); (3) dedicated energy crops (which are mostly composed of fast growing tall, woody grasses); and/or (4) municipal garden waste and paper waste. In a preferred embodiment, the lignocellulosic biomass feedstock is selected from the group consisting of woody low branching plants, gramineous plants, and herbage plants, or a combination thereof. In other embodiments, the lignocellulosic biomass feedstock includes, but is not limited to, C4 grasses, such as switch grass, cord grass, rye grass, miscanthus, or a combination thereof, or sugar cane bagasse, soybean stover, corn stover, rice straw, rice hulls, barley straw, corn cobs, wheat straw, oat hulls, corn fiber, recycled wood pulp fiber, sawdust, hardwood, or softwood, or a combination thereof. Further, the lignocellulosic feedstock can comprise cellulosic waste material such as, but not limited to, newsprint, cardboard, sawdust and the like. Lignocellulosic feedstock can comprise one species of fiber or alternatively, lignocellulosic feedstock can comprise a mixture of fibers that originate from different lignocellulosic feedstocks. Further, the lignocellulosic feedstock can comprise fresh lignocellulosic feedstock, partially dried feedstock, fully dried feedstock, or a combination thereof. Preferably, the lignocellulosic feedstock comprises fully dried feedstock. In some preferred embodiments, the lignocellulosic biomass feedstock includes materials from woody low branching plants, gramineous plants, and herbage plants.

The lignocellulosic biomass feedstock in this invention can be used directly for the conversion process. In another preferred embodiment, the lignocellulosic feedstock can comprise a fragmented feedstock. Fragmentation of the lignocellulosic feedstock can be performed according to any method known in the art provided that the method is capable of reducing the lignocellulosic feedstock into particles of an adequate size, for example, mechanical disruption, sonication, etc. For example, but not to be considered limiting, mechanical disruption of straw preferably results in pieces of straw having a length less than about 2.5 cm. Preferably, fragmentation of lignocellulosic feedstock produces particles that can pass through about 8-64 mesh filters. Without wishing to be limiting, mechanical disruption of lignocellulosic feedstock can be performed by chopping, chipping, grinding, milling, shredding or the like. Preferably, mechanical disruption is performed by milling, for example, but is not limited to, szego milling, hammer milling or wiley milling. However, the method of the present invention also contemplates the use of undisrupted lignocellulosic feedstock comprising a particle size which can pass through about 8-64 mesh filters.

In some embodiments, the acidic liquor used in the conversion processes of this invention comprises an acid with or without dilution. In other embodiments, the acidic liquor comprises a combination of acids with or without dilution. The concentration of acid(s) in the process can be between about 0.1% to about 100% (v/v), diluted in various solvents. In one preferred embodiment, the acid(s) is diluted in water. In some preferred embodiments, the concentration of the acid(s) is, for example, 0.1%, 0.2%, 0.5%, 1%, 2%, 4%, 5%, 6%, 8%, 10%, 20%, 30%, 40%, 50%, 60%, 70%, 80%, 90% or 100%. In some most preferred embodiments, the concentration of the acid(s) is, for example, 0.1%, 0.2%, 0.5%, 1%, 1.5%, 2%, 2.5%, 3%, 3.5%, 4%, 4.5%, or 5.0%. In other preferred embodiments, the molar concentration of the acid(s) is, for example, 0.1 mole/L, 0.2 mole/L, or 0.5 mole/L.

In some embodiments, the weight:volume (w/v) ratio of the biomass feedstock vs. the acidic liquor can be, for example, 1:2-1:20. In some embodiments, the biomass feedstock is incubated with the acidic liquor at moderate temperature. In one preferred embodiment, the moderate temperature is, for example, 70° C.-120° C. In one preferred embodiment, the biomass feedstock is incubated with 0.1%-5% dilute acid at 120° C.-160° C. In another preferred embodiment, the biomass feedstock is incubated with 10%-100% concentrated acid at 50° C.-110° C. In another preferred embodiment, the biomass feedstock is incubated with 0.1%-5% dilute acid at 50° C.-90° C., followed by an optional conditioning step at 120° C.-160° C. before grinding step (e.g., in step (v)). In some embodiments, the biomass is incubated with the acidic liquor for, for example, between about 1 to about 16 hours. In one preferred embodiment, the biomass feedstock is incubated with the acidic liquor under a moderate pressure that will not inhibit or prevent the conversion process. In one preferred embodiment, the biomass feedstock is incubated with the acidic liquor under a saturated vapor pressure of the temperature of acidic liquor. In one preferred embodiment, the pressure is normal atmospheric pressure. In another preferred embodiment, the pressure is approximately 1.0 MPa.

In a preferred embodiment, the acidic liquor comprises a low-boiling point organic acid. In another preferred embodiment, the acidic liquor comprises a combination of low-boiling point organic acids. In other embodiments, the acidic liquor can comprise other organic acids, inorganic acids, mineral acids, etc. In a preferred embodiment, the low-boiling point organic acid includes, for example, formic acid, acetic acid, 2-hydroxypropionic acid, propionic acid, acrylic acid, propylene-2-carboxylic acid, n-pentanoic acid, lactic acid, trifluoromethane sulfonic acid, methyl acrylic acid or trifluoroacetic acid (TFA), or a combination thereof. In a preferred embodiment, the low-boiling point organic acid is trifluoroacetic acid (TFA). The acid(s) used in the present invention can also include inorganic acid, mineral acids, or any acids known to a person with ordinary skills in the art, or a combination thereof. Examples include, but are not limited to, hydrochloric acid (HCl), sulfuric acid (H₂SO₄), phosphoric acid (H₃PO₄), and nitric acid (HNO₃).

In some embodiments, the biomass is incubated with the acidic liquor at a temperature between about 70° C. to about 150° C. In some embodiments, the biomass is incubated with the acidic liquor for a period of time between about 1 to about 16 hours. In one preferred embodiment, the biomass is incubated with 0.5% TFA at 90° C. for 16 hours. In another preferred embodiment, the biomass is incubated with 70% TFA at 90° C. for 5 hours. In one preferable embodiment, 0.2% (w/v) TFA is used to incubate with wheat straw at 90° C. for 16 hours at normal atmospheric pressure. In one preferable embodiment, 0.5% (w/v) TFA is used to incubate with wheat straw at 90° C. for 6 hours at normal atmospheric pressure. In another preferable embodiment, 0.2 mole/L nitric acid is used to incubate with corn stover at 90° C. for 5 hours at normal atmospheric pressure. In another preferable embodiment, 0.2 mole/L hydrochloric acid is used to incubate with corn stover at 90° C. for 5 hours at normal atmospheric pressure. In another preferable embodiment, 0.2 mole/L sulfuric acid is used to incubate with corn stover at 90° C. for 16 hours at normal atmospheric pressure. In another preferable embodiment, 0.2 mole/L phosphoric acid is used to incubate with corn stover at 90° C. for 16 hours at normal atmospheric pressure. In another preferable embodiment, 0.5 mole/L chloric acid is used to incubate wheat straw at 90° C. for 5 hours at normal atmospheric pressure. In another preferable embodiment, 0.5 mole/L nitric acid is used to incubate wheat straw at 90° C. for 5 hours at normal atmospheric pressure.

In a preferred embodiment, the extracted hemicellulose fraction dissolved in the acidic liquor contains mainly mono- and oligo-xylose (at least 90% purity in the acidic liquor), along with low content of galactose, arabinose, lactose, glucose and other natural compounds.

In one embodiment, the conversion product of biomass is collected from the soluble fraction, and the xylose polymer and/or xylose oligomer is separated. In another embodiment, separating the xylose polymer and/or xylose oligomer from the soluble fraction of biomass conversion product is through ethanol precipitation. In a preferred embodiment, the concentration of ethanol for precipitation of xylose polymer and/or xylose oligomer from the soluble fraction of biomass conversion is 30%-90% (v/v). In one embodiment, the polymerization degree of xylose polymer and/or xylose oligomer obtained is 1.3-5.6. In some preferred embodiment, the polymerization degree of xylose polymer and/or xylose oligomer obtained is 4.3-5.6.

In this invention, the incubation of the biomass with the acidic liquor produces the resulting acidic liquor and solid residue. The acidic liquor can then be separated from the resulting solid residue of the biomass using methods known to a person with ordinary skills in the art. The compositions of the resulting acidic liquor and solid residue can be used directly for various aims. In some embodiments, the acidic liquor is discarded. In one preferred embodiment, the acidic liquor is recovered for reuse. In one embodiment, the soluble product(s) of the biomass can be collected, recovered, purified, and/or concentrated from the acidic liquor. In another embodiment, the resulting solid residue can be heated for recovering the residual acidic liquor in the solid residue. In a preferred embodiment, the resulting solid residue is heated at a temperature higher than the temperature of the initial incubation in the conversion process. In a preferred embodiment, the resulting solid residue is heated at between about 70° C. to about 180° C. In another embodiment, the resulting solid residue is heated for between about 3 hours to about 6 hours. In another preferred embodiment, the residual acidic liquor in the solid residue is evaporated (using, e.g., a rotary evaporator) and then collected for recycling.

In one embodiment, the conversion product of biomass is collected from the soluble fraction. In one embodiment, the conversion product is collected from the insoluble fraction. In one embodiment, the conversion product is collected from both the soluble and the insoluble fractions. In one preferred embodiment, the soluble product(s) of the biomass is collected from the resulting acidic liquor. In one preferred embodiment, the soluble product(s) of the biomass is purified from the acidic liquor and concentrated. In another preferred embodiment, the solid residue is further processed to produce more products. These further processes include, for example, more rounds of incubation with the same acidic liquor under the same or different conditions, i.e., biomass/acidic liquor ratio, time, temperature, pressure, etc., more rounds of incubation under different conditions, i.e., biomass/acidic liquor ratio, time, temperature, pressure, component(s) of the acidic liquor, concentration(s) of acidic liquor, concentration(s) or ratio of component(s) of the acidic liquor, etc., and new steps of hydrolysis, e.g., enzyme hydrolysis. In a preferred embodiment, cellulase enzyme is used to further hydrolyze the resulting solid residues. In other embodiments, other enzymes or a combination of different enzymes are used for further hydrolysis. The solid residue can be directly used for enzyme hydrolysis. In other preferred embodiments, the solid residue is fragmented before the hydrolysis to improve its contact with the enzyme and thus the efficiency of the enzyme. In one preferred embodiment, the cellulose hydrolysis rate is improved more than 2.5 fold. In one preferred embodiment, most cellulose are hydrolyzed. Fragmentation of the solid residue can be performed according to any method known in the art provided that the method is capable of reducing the residue into fine particles of an adequate size, for example, mechanical disruption, sonication, etc. Without wishing to be limiting, mechanical disruption can be performed by chopping, chipping, grinding, milling, shredding or the like. Preferably, mechanical disruption is performed by grinding. In a preferred embodiment, grinding is used for the fragmentation step. In another preferred embodiment, conditioning of the insoluble solid residue at, for example, 120° C.-160° C., is used prior to the grinding.

In one preferred embodiment, the hydrolysis of the cellulose component of the biomass occurs before or in conjunction with the acid incubation process. In another preferred embodiment, the hydrolysis of the cellulose component of the biomass is after the acid incubation process. In one preferred embodiment, the cellulose hydrolysis comprises an incubation process of the grinded solid residue with cellulase enzymes at about 10° C. to about 90° C. In a preferred embodiment, the hydrolysis produces a soluble fraction comprising glucose. In a preferred embodiment, the purity of glucose in the soluble fraction is at least 90%. In a preferred embodiment, the hydrolysis produces an insoluble fraction comprising lignin. In a preferred embodiment, the purity of lignin in the insoluble fraction is at least 90%. In one preferred embodiment, the composition of lignin in the insoluble fraction is further processed to produce other biochemicals. In a preferred embodiment, the composition of lignin is further converted to produce other biochemicals under thermolysis. These biochemicals include, for example, aromatic chemicals, i.e., benzene, toluene, xylene derivatives and related chemicals, etc. In a preferred embodiment, these biochemicals include lignin-related products, for example, conferols, propylphenol, eugenol, syringols, aryl ethers, and alkylated methyl aryle ethers, as well as related compounds. In some embodiments, the composition of lignin can be heated directly to produce these chemicals. In other embodiments, lignin is further purified and/or concentrated from the composition before thermolysis. In some embodiments, the lignin composition is heated at about 300° C. to about 500° C. In other embodiments, the lignin is heated with or without catalyst. In a preferred embodiment, the lignin is heated at 450° C. under vacuum to produce a resulting liquid comprising at least 5 compounds with a total content of at least 75%. In another preferred embodiment, the lignin is mixed with Al₂O₃ and Fe₂O₃ as catalyst and heated at 400° C. under vacuum to produce a resulting liquid comprising at least 5 compounds with a total contents of at least 79%.

With the process(es) of this invention, different fractions of the biomass are separated for further purification or further conversions. Some exemplified soluble or insoluble products comprise cellulose, hemicellulose, xylose polymer, xylose oligomer, xylose monomer, glucose, lignin, and other lignocellulosic products, or a combination thereof. These products can be used directly in various areas. In some preferred embodiments, all these products produced in the continuous processes described above can be further used to culture microbes to be converted to bioenergy, biochemicals, or other bulk materials, or a combination thereof. Some non-limiting examples of these biochemicals and bulk materials include bioethanol, bio-diesel, citric acid, aspartic acid, lactic acid, amino acid, natural compounds, health care products and animal feedstuff, fragrant fine chemical & pharmaceutical and carbon fibre, ethanol, sorbitol, acetic acid, ascorbic acid, xylitol, propanediol, butanediol, acetone, butanol, benzene, toluene, and xylene derivatives, or a combination thereof. In a preferred embodiment, ethanol is the converted product after the continuous processes. The methods to further convert products to these biochemicals include, for example, microbial fermentation. In some embodiments, the fermentation of microbe culture is used for the conversion. In one preferred embodiment, the microbe is Pichia pastoris GS 115. In one embodiment, the microbe is a bacterial cell. In one preferred embodiment, the bacterial cell is originated from Escherichia coli. In another preferred embodiment, the bacterial cell is originated from Zymomonas mobilis. In another embodiment, the microbe is a yeast cell. In one preferred embodiment, the yeast cell is originated from the Saccharomyces species, for example, Saccharomyces bayanus, Saccharomyces carlsburgenesis or Saccharomyces cerevisiae. A particularly preferred microbial host is Saccharomyces cerevisiae. In yet another embodiment, the microbe is a fungus. In one preferred embodiment, the fungus is originated from the genus Paecilomyces. Other non-limiting examples for the microbe include members of the genera Methylococcus, Ralstonia, Aneurinibacillus, Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Preferred hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, Saccharomyces carlsburgenesis and Saccharomyces cerevisiae. In some embodiments, the microbe is genetically modified to express an exogenous protein beneficial for the ethanol production. In one embodiment, the exogenous protein has a function in the ethanol-producing pathway in the microbe. In another embodiment, the exogenous protein is an enzyme. In one preferred embodiment, the enzyme is pyruvate decarboxylase. In another preferred embodiment, the enzyme is alcohol dehydrogenase.

This invention also provides information to prepare various lignocellulosic originated compositions produced by the processes discussed above. In one preferred embodiment, a lignocellulosic originated composition comprises xylose polymer, xylose oligomer, xylose monomer, or a combination thereof, produced by the incubation of the biomass with the acidic liquor. In another preferred embodiment, a lignocellulosic originated composition comprises glucose, produced by the further processing, i.e., hydrolysis, of the solid residue from the incubation of the biomass with the acidic liquor. In another preferred embodiment, a lignocellulosic originated composition comprises lignin, produced in the insoluble fraction of the solid residue the incubation of the biomass with the acidic liquor. All these compositions discussed above contain a high content of the lignocellulosic originated products. In one preferred embodiment, the content is at least 90%.

This invention also provides a lignocellulosic feedstock processing system comprising a set of devices capable of carrying the incubation, separation, or further processings, or a combination of these functions, for processing of lignocellulosic feedstock. In a preferred embodiment, the lignocellulosic feedstock processing system further comprises a feedstock handling device and a preconditioner capable of receiving the lignocellulosic feedstock, or previously fragmented feedstock, from the handling device, while the preconditioner is also in communication with set of devices of processing system.

One non-limiting example of culturing at least one species of microbes is the following protocol for the culture of Pichia pastoris GS115 under a pH of 6.85: A seed culture is grown by inoculating 50 mL M9 minimal broth (contained 6 g/L Na₂HPO₄, 3 g/L KH₂PO₄, 0.5 g/L NaCl, 1 g/L NH₄Cl, plus 20% glucose with a single colony of Pichia pastoris GS115 in a 250-mL flask and incubating at 30° C. with shaking at 165 rpm. After 10 hours of growth, 1 mL of the seed culture is centrifuged at 8,000 rpm. Cell pellet is resuspended in 200 μL distilled water, inoculated on the M9 minimal plate (1.8% agar) plus the supernatant (4% reducing sugar) and cultured at 30° C. for seven days. The clone size on agar plate and cell density in the broth are measured as the growth condition.

The term “lignocellulosic biomass feedstock”, or “lignocellulosic feedstock”, refers to material accumulated during plant growth, including but not limited to, 1) forest wood and branches including logging residues, rough, rotten, and salvable dead wood, excess saplings, small pole trees, aquatic plants, wood wastes and residues; 2) agricultural food and feed crops and their residues; 3) herbaceous and woody energy crops and their residues; and 4) other waste materials including municipal wastes, which can be, for example, hardwood, softwood, recycled paper, waste paper, forest trimmings, pulp and paper waste, corn stover, corn fiber, wheat straw, rice straw, sugarcane bagasse, or switchgrass.

The term “low boiling point” refers to a boiling point not higher than 160° C. at normal atmospheric pressure.

The term “organic acid” refers to acid that can be refined through distillation or sublimation, including but not limited to, formic acid, acetic acid, trifloroacetic acid, difluoroacetic acid, monofluoroacetic acid, propionic acid, butyric acid, trifluoromethane sulfonic acid, methanesulfonic acid, glycolic acid, DL-lactic acid, n-butyric acid, and mercaptoacetic acid, or a combination thereof. In a preferred embodiment, low boiling point organic acid represents optionally trifluoroacetic acid, formic acid, acetic acid, propionic acid, or lactic acid.

The term “TFA” refers to trifloroacetic acid.

The term “soluble extracts” refers to materials dissolvable in the acid liquor of concentration of 0.5-100% and precipitated from the filtrate after the removal of the acid and water.

The term “fine chemicals & pharmaceuticals of natural origin” refers to the natural compounds of physiological activity synthesized during plant growth and remained in the feedstock; The term “proteins & amino acids” refers to the proteins and amino acids contained in the biomass feedstock, and the peptide and amino acids degraded from those proteins.

Throughout the specification and claims the term “grinding” refers to any method of reducing a size of a solid, such as, but not limited to, grinding, pulverizing, milling, disintegrating, rubbing, granulating, rasping, crushing, grating, dashing, breaking, etc. In one embodiment, the solid is grinded into fine particles.

The invention will now be described in further detail with reference to the following examples. The examples are provided for illustrative purposes, and are not to be construed as limiting the scope of the invention in any way.

### EXAMPLES

Throughout the examples, the following methods are used unless otherwise stated.

### Methods

The content analysis of cellulose, hemicellulose and lignin in biomass feedstock and solid intermediate residues is carried out according to NREL Chemical Analysis and Testing Laboratory Analytical Procedures (LAP's) LAP-002 of Determination of Carbohydrates in Biomass by High Performance Liquid Chromatography, LAP-003 of Determination of Acid-Insoluble Lignin in Biomass and LAP-004 of Determination of Acid-Soluble Lignin in Biomass. These protocols can be found in the National Renewable Energy Laboratory (http://www.nrel.gov/biomas s/analytical_procedures.html; http://cobweb.ecn.purdue.edu/˜lorre/16/research/). The cellulase efficiency assay of cellulose hydrolysis of solid intermediate residues is carried out according to NREL Chemical Analysis and Testing Laboratory Analytical Procedures (LAP's) LAP-006 of Measurement of Cellulase Activities.

The definition of the “easiness of grinding” in the present invention is defined as: If it takes more than 10 minutes to grind the biomass solid residue in a mortar into fine particles, the degree of easiness of grinding is denoted as “+”. If it takes less than 10 seconds to grind such solid residue into fine particles, the degree of easiness of grinding is denoted “+++++”.

### Example 1

### Experiments 1 to 8

Wheat straw is dried at 50° C. and cut into fragments 2.5 cm in length. 1.0 g straw fragments and 10 ml of trifluoroacetic acid (TFA) at a concentration of 30%-100% are added to a hydrothermal reaction vessel, sealed, and heated at 80° C. for 3 hours. The resulting reaction mixture is cooled to room temperature and then filtered. The resulting filtration solid residue is dried at 50° C. for carbohydrate and lignin analysis. The remaining TFA in the filtration is evaporated with a rotary evaporator before a reducing sugar analysis of the resulting filtration solid residue.

In Table 1, the initial concentration of trifluoroacetic acid (TFA), the reducing sugar yield in supernatant, the amount of solid residue mass, and the carbohydrates and lignin content of the residue are shown for Experiments 1 to 8.

TABLE 1
Experiments 1 to 8

| | | Reducing | | | | |
|---|---|---|---|---|---|---|
| | | sugar yield | | | | |
| | TFA | in | Solid residue | | | |
| | conc. | supernatant | Solid | Carbohydrates | Acid insoluble | Soluble |
| Experiments | (%) | (g) | mass (g) | (%) | lignin (%) | lignin (%) |
| 1 | 30% | 0.26 | 0.76 | 52 | 34 | 1.8 |
| 2 | 40% | 0.28 | 0.72 | 59 | 20 | 1.9 |
| 3 | 50% | 0.31 | 0.68 | 64 | 21 | 1.8 |
| 4 | 60% | 0.33 | 0.62 | 64 | 23 | 1.8 |
| 5 | 70% | 0.31 | 0.56 | 67 | 23 | 1.9 |
| 6 | 80% | 0.29 | 0.55 | 65 | 24 | 1.5 |
| 7 | 90% | 0.27 | 0.49 | 63 | 28 | 1.1 |
| 8 | 100% | 0.21 | 0.19 | 51 | 40 | 1.1 |

### Example 2

### Experiments 9 to 16

Wheat straw is dried at 50° C. and cut into 2.5 cm fragments. 1.0 g straw fragments and 10 ml of 75% trifluoroacetic acid (TFA) of different concentration are added to a hydrothermal reaction vessel, sealed, and heated at 80° C. for 2 to 24 hours. The resulting reaction mixture is cooled to room temperature and then filtered. The resulting filtration solid residue is dried at 50° C. for carbohydrate and lignin analysis. The remaining TFA in the filtration is evaporated with a rotary evaporator before a reducing sugar analysis of the resulting filtration solid residue.

In Table 2, the initial concentration of trifluoroacetic acid, the reducing sugar yield in supernatant, and the amount of solid residue mass, the carbohydrates and lignin content of the residue are shown for Experiments 9 to 16.

TABLE 2
Experiments 9 to 16

| | | Reducing | | | | |
|---|---|---|---|---|---|---|
| | | sugar yield | Solid residue | | | |
| | | in | | | Acid- | |
| | Time | supernatant | Solid | Carbohydrates | insoluble | Soluble |
| Experiments | (hours) | (g) | mass (g) | (%) | lignin (%) | lignin (%) |
| 9 | 2 | 0.24 | 0.60 | 60 | 23 | 0.8 |
| 10 | 3 | 0.28 | 0.59 | 64 | 20 | 0.8 |
| 11 | 4 | 0.29 | 0.56 | 64 | 22 | 0.7 |
| 12 | 5 | 0.32 | 0.57 | 64 | 20 | 0.7 |
| 13 | 6 | 0.29 | 0.54 | 62 | 19 | 0.9 |
| 14 | 7 | 0.29 | 0.53 | 64 | 18 | 0.7 |
| 15 | 12 | 0.25 | 0.54 | 62 | 26 | 0.5 |
| 16 | 24 | 0.19 | 0.53 | 55 | 31 | 0.6 |

### Example 3

### Experiments 17 to 25

Wheat straw is dried at 50° C. and cut into 2.5 cm fragments. 1.0 g straw fragments and 10 ml of 75% trifluoroacetic acid (TFA) of different concentration are added to a hydrothermal reaction vessel, sealed, and heated at 70° C. to 150° C. for 3 hours. The resulting reaction mixture is cooled to room temperature and then filtered. The resulting filtration solid residue is dried at 50° C. for carbohydrate and lignin analysis. The remaining TFA in the filtration is evaporated with a rotary evaporator before a reducing sugar analysis of the resulting filtration solid residue.

In Table 3, the initial concentration of trifluoroacetic acid, the reducing sugar yield in supernatant, and the amount of solid residue mass, the carbohydrates and lignin content of the residue are shown for Experiments 17 to 25.

TABLE 3
Experiments 17 to 25

| | | Reducing sugar | Solid residue | | | |
|---|---|---|---|---|---|---|
| | | yield in | Solid | | | Soluble |
| | Temperature | supernatant | mass | Carbohydrate | Acid-insoluble | lignin |
| Experiments | (° C.) | (g) | (g) | (%) | lignin (%) | (%) |
| 17 | 70 | 0.27 | 0.70 | 68 | 21 | 0.8 |
| 18 | 80 | 0.23 | 0.63 | 70 | 22 | 0.6 |
| 19 | 90 | 0.22 | 0.61 | 75 | 17 | 0.6 |
| 20 | 100 | 0.22 | 0.56 | 68 | 20 | 0.6 |
| 21 | 110 | 0.19 | 0.54 | 71 | 20 | 0.5 |
| 22 | 120 | 0.18 | 0.54 | 75 | 26 | 0.6 |
| 23 | 130 | 0.07 | 0.48 | 72 | 26 | 0.4 |
| 24 | 140 | 0.08 | 0.44 | 66 | 28 | 0.4 |
| 25 | 150 | 0.08 | 0.33 | 14 | 65 | 0.4 |

### Example 4

### Experiments 26 to 32

Wheat straw is dried at 50° C. and cut into 2.5 cm fragments. 1.0 g feedstock fragments and 10 ml trifluoroacetic acid are added to a hydrothermal reaction vessel, sealed, and heated at 90° C. for different hours. The resulting reaction mixture is cooled to room temperature and then filtered. The remaining TFA in the filtration is evaporated with a rotary evaporator before a reducing sugar analysis and a total carbohydrate analysis of the resulting filtration solid residue.

In Table 4, the initial trifluoroacetic acid concentration and incubation time, the reducing sugar yield, the total soluble carbohydrate yield, and average degree of polymerization of the soluble carbohydrate are shown for Experiments 26 to 32. The peak total soluble carbohydrate yield is about 30% under conditions in which the sample is heated at 90° C. with 1% TFA for 5 hours or with 0.5% TFA for 16 hours.

The average degree of polymerization of the soluble carbohydrate is 17.2, 7.3, 3.1 or 2.5 under conditions in which the sample is heated with 1% of trifluoroacetic acid for 1, 2, 3 or 4 hours, respectively.

TABLE 4
Experiments 26 to 32

| | | | Total soluble | Average degree of |
|---|---|---|---|---|
| | Acid concentration | Reducing sugar | carbohydrate | polymerization of |
| Experiments | and incubation time | yield (%) | yield (%) | soluble carbohydrate |
| 26 | 1% for 1 hours | 1.0% | 17.2% | 17.2 |
| 27 | 1% for 2 hours | 2.9% | 21.2% | 7.3 |
| 28 | 1% for 4 hours | 7.7% | 23.8% | 3.1 |
| 29 | 1% for 5 hours | 11.8% | 29.5% | 2.5 |
| 30 | 0.1% for 16 hours | 21.4% | 24.0% | 1.1 |
| 31 | 0.2% for 16 hours | 25.1% | 26.5% | 1.1 |
| 32 | 0.5% for 16 hours | 28.0% | 29.4% | 1.1 |

### Example 5

### Experiments 33 to 39

Wheat straw is dried at 50° C. and cut into 2.5 cm fragments. 1.0 g feedstock fragments and 10 ml different type acid are added to a hydrothermal reaction vessel, sealed, and heated at 90° C. for different hours. The resulting reaction mixture is cooled to room temperature and then filtered. The remaining TFA in the filtration is evaporated with a rotary evaporator before a reducing sugar analysis and a total carbohydrate analysis of the resulting filtration solid residue.

In Table 5, the type and initial concentration of acid, and the total soluble carbohydrate yield in supernatant are shown for Experiments 33 to 39.

TABLE 5
Experiments 33 to 39

| | | Acid concentration | Total reducing sugar yield |
|---|---|---|---|
| Experiments | Acid type | and incubation time | in supernatant (%) |
| 33 | Formic Acid | 9% for 5 hours | 10.3% |
| 34 | Acetic Acid | 15% for 5 hours | 22.8% |
| 35 | 2-Hydroxypropionic acid | 10% for 5 hours | 21.5% |
| 36 | Propionic acid | 3% for 5 hours | 16.8% |
| 37 | Acrylic acid | 7% for 5 hours | 13.6% |
| 38 | Propylene-2-carboxylic | 4% for 5 hours | 13.1% |
| | acid | | |
| 39 | n-Pentanoic acid | 5% for 5 hours | 8.7% |

### Example 6

### Experiments 40 to 46

Wheat straw is dried at 50° C. and cut into 2.5 cm fragments. 1.0 g feedstock fragments and 10 ml different type acid are added to a hydrothermal reaction vessel, sealed, and heated at 160° C. for 24 hours. The resulting reaction mixture is cooled to room temperature and then filtered. The resulting filtration solid residue is dried at 50° C. for carbohydrate and lignin analysis. The remaining TFA in the filtration is evaporated with a rotary evaporator before a reducing sugar analysis of the resulting filtration solid residue.

In Table 6, the type of acid and initial concentration, the reducing sugar yield in supernatant, and the carbohydrates and lignin content of the solid residue are shown for Experiments 40 to 46.

TABLE 6
Experiments 40 to 46

| | | Reducing | Solid residue | | |
|---|---|---|---|---|---|
| | | sugar yield in | | Acid | |
| | | Supernatant | Carbohydrates | insoluble | Soluble |
| Experiments | Acid type | (g) | (%) | lignin (%) | lignin (%) |
| 40 | Formic acid | 0.064 | 37 | 15 | 0.59 |
| | (100%) | | | | |
| 41 | Acetic acid | 0.150 | 88 | 23 | 0.91 |
| | (100%) | | | | |
| 42 | Lactic acid | 0.169 | 77 | 29 | 0.89 |
| | (100%) | | | | |
| 43 | Trifluoromethanesulfonic | 0.178 | 20 | 25 | 0.48 |
| | acid (10%) | | | | |
| 44 | Propionic acid | 0.120 | 25 | 8 | 0.54 |
| | (100%) | | | | |
| 45 | Methyl acrylic | 0.095 | 40 | 19 | 0.51 |
| | acid (100%) | | | | |
| 46 | Pentanoic acid | 0.141 | 38 | 11 | 0.30 |
| | (100%) | | | | |

### Example 7

### Experiments 47 to 55

Different feedstocks are dried at 50° C. and cut into 2.5 cm fragments. 1.0 g feedstock fragments and 10 ml of 75% trifluoroacetic acid (TFA) are added to a hydrothermal reaction vessel, sealed, and heated at 70° C. for 3 hours. The resulting reaction mixture is cooled to room temperature and then filtered. The resulting filtration solid residue is dried at 50° C. for carbohydrate and lignin analysis. The remaining TFA in the filtration is evaporated with a rotary evaporator before a reducing sugar analysis of the resulting filtration solid residue.

In Table 7, the initial concentration of trifluoroacetic acid, the reducing sugar yield in supernatant, the amount of solid residue mass, and the carbohydrates and lignin content of the residue are shown for Experiments 47 to 55.

TABLE 7
Experiments 47 to 55

| | | Reducing | | | |
|---|---|---|---|---|---|
| | | sugar yield in | Solid residue | | |
| | | supernatant | Carbohydrates | Acid insoluble | Soluble lignin |
| Experiments | Feedstock | (g) | (%) | lignin (%) | (%) |
| 47 | Cotton stalk | 0.20 | 67 | 28 | 1.1 |
| 48 | Pine wood | 0.21 | 70 | 40 | 0.4 |
| 49 | Cynodon | 0.29 | 63 | 34 | 1.7 |
| | dactylon | | | | |
| | (L.) Pers | | | | |
| 50 | Miscanthus | 0.20 | 73 | 20 | 1.4 |
| | sinensis | | | | |
| 51 | Bamboo | 0.19 | 66 | 38 | 0.8 |
| 52 | Reed | 0.23 | 77 | 28 | 0.9 |
| 53 | Eulaliopsis | 0.25 | 79 | 16 | 1.1 |
| | binata | | | | |
| | (Retz.) C.E. | | | | |
| | Hubb. | | | | |
| | sabaigrass | | | | |
| 54 | Wheat | 0.26 | 70 | 25 | 0.9 |
| | straw | | | | |
| 55 | Cotton cob | 0.40 | 79 | 21 | 1.3 |

### Example 8

### Experiments 56 to 65

The filtration solid residue from Experiments 33 to 41 is dried at 50° C. 0.1 g of dried residue is taken and wetted with 100 ml of acetic acid-sodium acetate buffer (0.01 M, pH 4.6), grinded fine, and incubated with 5.6 FPU cellulase (Fibrilase HDL160, Iogen, Canada) at 50° C. with shaking at 165 rpm. The microcrystal cellulose Avicel PH105 (from Serva) is used as control. Then aliquots of reaction mixture are taken after 0.5, 1, 2, 4, 6, 8, 10, 12, and 24 hour and assayed with DNS reducing sugar assay to calculate the reducing sugar yield and the time taken for 80% carbohydrate hydrolysis. At 24 hours, the mixture is filtrated, and filtration solid residue is dried at 50° C., weighed and analyzed for lignin and carbohydrates content.

In Table 8, the initial total insoluble carbohydrates, the time taken for 80% insoluble carbohydrates hydrolysis, the reducing sugar yield at 24 hours, the solid lignin residue recovered at 24 hours, and the carbohydrates and lignin content in the solid lignin residue are shown for Experiments 56 to 64. The time taken for 80% insoluble carbohydrate hydrolysis is an indicator of insoluble carbohydrate hydrolysis efficiency into glucose with same usage of cellulase enzyme. The un-grinded wheat straw takes about 40 hours to reach 50% hydrolysis, the highest hydrolysis is around 60% (after 60 hours). However, in the present invention, as shown in Table 8, it takes only 0.8 hour for processed wheat straw residue to reach 80% insoluble carbohydrate hydrolysis, about 0.9 hour and 1.4 hours for processed cotton stalk and cotton cob residues to reach 80% insoluble carbohydrate hydrolysis, respectively. As a comparison, It takes about 13.5 hours for microcrystal cellulose Avicel PH105 requires to achieve 80% hydrolysis.

TABLE 8
Experiments 56 to 65

| | | Initial total | Time for 80% | | Solid lignin residue | | | |
|---|---|---|---|---|---|---|---|---|
| | | insoluble | carbohydrate | Reducing | Solid | | Acid | |
| | Type of | carbohydrates | hydrolysis | sugar yield at | mass | Carbohydrates | insoluble | Soluble |
| Experiments | Feedstock | (mg) | (hour) | 24 hour (mg) | yield (g) | (%) | lignin (%) | lignin (%) |
| 56 | Cotton stalk | 67 | 0.9 | 71.5 | 0.035 | 7 | 94 | 0.17 |
| 57 | Pine wood | 70 | 6.2 | 66.2 | 0.035 | 11 | 96 | 0.09 |
| 58 | Cynodon | 63 | 1.8 | 67.2 | 0.040 | 5 | 95 | 0.16 |
| | dactylon (L.) | | | | | | | |
| | Pers | | | | | | | |
| 59 | Miscanthus | 73 | 3.3 | 73.5 | 0.031 | 2 | 96 | 0.17 |
| | sinensis | | | | | | | |
| 60 | Bamboo | 66 | 3.2 | 66.6 | 0.039 | 4 | 98 | 0.11 |
| 61 | Reed | 77 | 6 | 80.9 | 0.026 | 3 | 97 | 0.16 |
| 62 | Eulaliopsis | 79 | 9.2 | 79.7 | 0.025 | 3 | 95 | 0.17 |
| | binata | | | | | | | |
| | (Retz.) C.E. | | | | | | | |
| | Hubb. | | | | | | | |
| | sabaigrass | | | | | | | |
| 63 | Wheat straw | 70 | 0.8 | 74.6 | 0.033 | 3 | 96 | 0.15 |
| 64 | Cotton cob | 79 | 1.4 | 81 | 0.024 | 6 | 95 | 0.23 |
| 65 | Avicel | | 13.5 | | | | | |
| | PH105 | | | | | | | |

### Example 9

### Experiment 66

For Experiment 66, wheat straw is dried at 50° C. and cut into 2.5 cm fragments. 10 duplicates of 1.0 g feedstock fragments and 10 ml of 70% trifluoroacetic acid (TFA) are added to a hydrothermal reaction vessel, sealed, and heated at 90° C. for 5 hours. The resulting reaction mixture is cooled to room temperature and then filtered. The resulting filtration solid residue is dried at 50° C. for carbohydrate and lignin analysis. As the result, the content of carbohydrates, acid-insoluble lignin, and acid-soluble lignin are 70%, 25%, and 0.9%, respectively. The remaining TFA in the filtration is evaporated with a rotary evaporator before a reducing sugar analysis of the resulting filtration solid residue. As the result, the reducing sugar is of 0.26 g. The HPLC analysis shows that the reducing sugar contains mainly xylose, whose content is about 92.5%±1.3%.

The solid residue is dried at 50° C. Then 0.1 g of dried residue is taken and mixed with an acetic acid-sodium acetate buffer (0.01M, pH4.6), after grinding fine, was incubated with ˜5.0 FPU cellulase (Accellerase™ 1000, Genencor, USA) at 50° C. with shaking at 165 rpm. Aliquots of mixture are taken at 1, 2, 4, 8, 24 hour for DNS reducing sugar and total carbohydrate analysis. As a result, the hydrolysis time for 80% carbohydrate is 1.7 hours. Further, the final carbohydrate hydrolysis yield is about 96%, corresponding to the reducing sugar yield of 74 mg. The HPLC analysis shows that the reducing sugar is mainly glucose with a content about 93.9%±1.2%. The remaining lignin residue is collected, dried at 50° C., and weighed to be 33 mg. For carbohydrate and lignin analysis, the content of carbohydrates, acid-insoluble lignin and acid-soluble lignin are 3%, 96% and 0.15%, respectively.

A seed culture of Pichia pastoris GS115 is grown by inoculating 50 mL M9 minimal broth plus 20% glucose with a single colony of Pichia pastoris GS115 in a 250-mL flask and incubating at 30° C. with shaking at 165 rpm. After 10 hours of growth, 1 mL of the seed culture is centrifuged at 8,000 rpm. Cell pellet is resuspended in 200 μL distilled water, inoculated on the M9 minimal plate (1.8% agar) plus a solution of 4% xylose fraction obtained in first step of Experiments 66 with the pH adjusted to 6.85, and cultured at 30° C. for seven days. The M9 minimal broth contained 6 g/L Na₂HPO₄, 3 g/L KH₂PO₄, 0.5 g/L NaCl, and 1 g/L NH₄Cl. The result shows that Pichia pastoris GS115 grows with prepared xylose as its carbon source.

### Example 10

### Experiments 67-92

Wheat straw is dried at 50° C. and cut into fragments less than 2.5 cm in length. 4 aliquots of 1.0 g dried fragments are taken and mixed with 1.0% trifluoroacetic acid (TFA), and heated at 90° C. for 1, 2, 3, 4 hours in a hydrothermal reaction vessel. The reaction mixture is cooled to room temperature and then filtered. Filtrations are further used for DNS reducing sugar and total carbohydrate analysis. The result shows that 1% TFA at 90° C. for 4 hours leads to a total soluble carbohydrate yield of 29%, suggesting that most hemicelluloses are hydrolyzed.

In Table 9, the initial concentration of trifluoroacetic acid, the reducing sugar yield in the supernatant, and the total soluble carbohydrate yield are shown for Experiments 67 to 92. The peak total soluble carbohydrate yield is about 30% under conditions in which the sample is heated with 1% TFA at 90° C. for 5 hours, at 110° C. for 4 hours, or at 120° C. for 1 hour.

TABLE 9
Experiments 67 to 92

| | | | Total soluble |
|---|---|---|---|
| | Incubation | Reducing sugar | carbohydrate |
| Experiments | condition | yield (%) | yield (%) |
| 67 | 90° C. for 1 hour | 1% | 13% |
| 68 | 90° C. for 2 hours | 3% | 16% |
| 69 | 90° C. for 3 hours | 4% | 19% |
| 70 | 90° C. for 4 hours | 5% | 27% |
| 71 | 90° C. for 5 hours | 8% | 29% |
| 72 | 90° C. for 6 hours | 9% | 30% |
| 73 | 90° C. for 7 hours | 10% | 30% |
| 74 | 90° C. for 8 hours | 10% | 30% |
| 75 | 110° C. for 1 hour | 1% | 17% |
| 76 | 110° C. for 2 hours | 7% | 22% |
| 77 | 110° C. for 3 hours | 8% | 25% |
| 78 | 110° C. for 4 hours | 26% | 30% |
| 79 | 110° C. for 5 hours | 26% | 29% |
| 80 | 110° C. for 6 hours | 26% | 30% |
| 81 | 110° C. for 7 hours | 28% | 30% |
| 82 | 110° C. for 8 hours | 28% | 30% |
| 83 | 120° C. for 1 hour | 23% | 29% |
| 84 | 120° C. for 2 hours | 22% | 28% |
| 85 | 120° C. for 3 hours | 19% | 27% |
| 86 | 120° C. for 4 hours | 19% | 30% |
| 87 | 120° C. for 5 hours | 17% | 28% |
| 88 | 140° C. for 1 hour | 11% | 23% |
| 89 | 140° C. for 2 hours | 14% | 26% |
| 90 | 140° C. for 3 hours | 19% | 30% |
| 91 | 140° C. for 4 hours | 24% | 30% |
| 92 | 140° C. for 5 hours | 26% | 29% |

### Example 11

### Experiments 93-95

Wheat straw is dried at 50° C. and cut into fragments less than 2.5 cm in length. 4 aliquots of 1.0 g are taken and mixed with 0.1, 0.2, or 0.5% trifluoroacetic acid (TFA), and heated at 90° C. for 16 hours in a hydrothermal reaction vessel. The reaction mixture is cooled to room temperature and then filtered. Filtrations are further used for DNS reducing sugar and total carbohydrate analysis. In Table 10, the initial concentration of trifluoroacetic acid and the total soluble carbohydrate yield in filtration are shown for Experiments 93 to 95. As described below, heating with 0.1% TFA at 90° C. for 16 hours can produce 24% total soluble carbohydrate yield. Considering the best practically achievable reduction of sugar yield is around 30% of feedstock (under mild conditions and long enough incubation time), the TFA hydrolysis yields in Experiments 93-95 are very high (more than 80% of hemicelluloses in the biomass feedstock are hydrolyzed).

TABLE 10
Experiments 93 to 95

| | Trifluoroacetic | |
|---|---|---|
| Experiments | acid (%) | Total soluble carbohydrate yield (%) |
| 93 | 0.1 | 24% |
| 94 | 0.2 | 26% |
| 95 | 0.5 | 28% |

### Example 12

### Experiments 96-98

Wheat straw, sugarcane bagasse, and cotton cob are dried at 50° C. and cut into fragments less than 2.5 cm in length. Aliquots of 300 g of each are taken and mixed with 0.5% trifluoroacetic acid (TFA) and heated at 90° C. for 16 hours in a hydrothermal reaction vessel. The reaction mixture is cooled to room temperature and then filtered. Filtrations are further dried with rotatory evaporator and the content of total soluble carbohydrate is then measured. As the result, the total soluble carbohydrate yield is 84 g from wheat straw (Experiment 96), 78 g from sugarcane bagasse (Experiment 97), and 108 g from cotton cob (Experiment 98).

### Example 13

### Experiments 99-134

0.2 g of residue wheat straw from Experiments 71, 94, or 95, with residue trifluoroacetic acid of 0.2%, 0.5%, or 1.0%, respectively, is heated in a hydrothermal reaction vessel at 120, 140 or 160° C. for 1-7 hour(s) (Experiments 99-127). After cooling to room temperature, samples of each residue wheat straw are taken and put in mortar to test the degree of easiness to grind into fine fragments.

Wheat straw is dried at 50° C. and cut into fragments less than 2.5 cm in length. Aliquots of 1.0 g of each are taken and mixed with 2, 3, 4, 5, 10, or 20% trifluoroacetic acid (TFA), and heated at 140° C. for 5 hours in a hydrothermal reaction vessel (Experiments 128-133). One allocation is treated with 70% trifluoroacetic acid (TFA) (Experiment 134), and heated at 90° C. for 5 hours in a hydrothermal reaction vessel. After cooling to room temperature, the residue wheat straw is put in mortar to test the degree of easiness to grind into fine fragments.

Definition of easiness of grinding: If it takes more than 10 minutes to grind the biomass fragments in a mortar into fine particles, the degree of easiness of grinding is denoted as “+”. If it takes less than 10 seconds to grind such fragments into fine particles, the degree of easiness of grinding is denoted “+++++”.

In Table 11, the degree of easiness of grinding, the concentration of trifluoroacetic acid, and the temperature/time of heating are shown for Experiments 99 to 134. As shown, the biomass solid residue can be grinded easily into fine particle when heated with 0.2% TFA to 140° C. for 6 hours, with 0.2% TFA to 160° C. for 5 hours, with 0.5% TFA to 140° C. for 5 hours, with 0.5% TFA to 160° C. for 3 hours, with 1.0% TFA to 120° C. for 6 hours, with 1.0% TFA to 140° C. for 4-6 hours, with 1.0-20% TFA to 140° C. for 4-6 hours and with 70% TFA to 90° C. for 5 hours.

TABLE 11
Experiments 99 to 134

| | TFA | | | Easiness of |
|---|---|---|---|---|
| Experiments | concentration | Temperature | Time | grinding |
| 99 | 0.2% TFA | 140° C. | 1 hour | + |
| 100 | 0.2% TFA | 140° C. | 2 hours | + |
| 101 | 0.2% TFA | 140° C. | 3 hours | + |
| 102 | 0.2% TFA | 140° C. | 4 hours | ++ |
| 103 | 0.2% TFA | 140° C. | 5 hours | ++ |
| 104 | 0.2% TFA | 140° C. | 6 hours | +++ |
| 105 | 0.2% TFA | 140° C. | 7 hours | +++ |
| 106 | 0.2% TFA | 160° C. | 1 hour | + |
| 107 | 0.2% TFA | 160° C. | 2 hours | ++ |
| 108 | 0.2% TFA | 160° C. | 3 hours | +++ |
| 109 | 0.2% TFA | 160° C. | 4 hours | +++ |
| 110 | 0.2% TFA | 160° C. | 5 hours | ++++ |
| 111 | 0.5% TFA | 140° C. | 1 hour | + |
| 112 | 0.5% TFA | 140° C. | 2 hours | + |
| 113 | 0.5% TFA | 140° C. | 3 hours | ++ |
| 114 | 0.5% TFA | 140° C. | 4 hours | ++ |
| 115 | 0.5% TFA | 140° C. | 5 hours | +++ |
| 116 | 0.5% TFA | 140° C. | 6 hours | +++ |
| 117 | 0.5% TFA | 140° C. | 7 hours | ++++ |
| 118 | 0.5% TFA | 160° C. | 1 hour | ++ |
| 119 | 0.5% TFA | 160° C. | 2 hours | +++ |
| 120 | 0.5% TFA | 160° C. | 3 hours | ++++ |
| 121 | 1% TFA | 120° C. | 5 hours | +++ |
| 122 | 1% TFA | 120° C. | 6 hours | +++ |
| 123 | 1% TFA | 140° C. | 1 hour | + |
| 124 | 1% TFA | 140° C. | 2 hours | + |
| 125 | 1% TFA | 140° C. | 3 hours | ++ |
| 126 | 1% TFA | 140° C. | 4 hours | ++++ |
| 127 | 1% TFA | 140° C. | 5 hours | +++++ |
| 128 | 2% TFA | 140° C. | 5 hours | ++++ |
| 129 | 3% TFA | 140° C. | 5 hours | ++++ |
| 130 | 4% TFA | 140° C. | 5 hours | ++++ |
| 131 | 5% TFA | 140° C. | 5 hours | ++++ |
| 132 | 10% TFA | 140° C. | 5 hours | ++++ |
| 133 | 20% TFA | 140° C. | 5 hours | ++++ |
| 134 | 70% TFA | 90 | 5 hours | +++++ |

### Example 14

### Experiments 135-140

15 mg lignin with purity at least 90% from Experiment 66 is allocated into glass ampoules, mixed with or without 3 mg of catalyst Al₂O₃ and Fe₂O₃ (w/w=50:1), sealed under vacuum and heated at 300° C.-450° C. for 12 hours. The thermolysis products are analyzed with GC-MS.

In Table 12, catalyst, temperature, thermolysis phenomenon, and the concentration of top 5 compounds are shown for Experiments 135 to 140.

TABLE 12
Experiments 135 to 140

| | | | | | Top 5 |
|---|---|---|---|---|---|
| | | | | Top 5 | compounds |
| | | | | compounds | contents in |
| Experiments | Catalyst | Temperature | Phenomenon | contents | total |
| 135 | — | 350° C. | Black powder, | 12.5%, 9.1%, | 44.2% |
| | | | clean liquid | 8.9%, 7.7%, | |
| | | | | 6.0% | |
| 136 | — | 400° C. | Black powder, | 19.9%, 15.2%, | 53.5% |
| | | | clean liquid | 7.1%, 6.8%, | |
| | | | | 4.4% | |
| 137 | — | 450° C. | Black powder, | 24.0%, 18.2%, | 75.4% |
| | | | clean liquid | 15.1%, 10.4%, | |
| | | | | 7.7% | |
| 138 | 3 mg | 300° C. | Black powder, | 14.4%, 7.9%, | 38.0% |
| | Al₂O₃ and | | more clean | 6.5%, 5.0%, | |
| | Fe₂O₃ | | liquid | 4.2% | |
| 139 | 3 mg | 350° C. | Black powder, | 10.0%, 6.9%, | 34% |
| | Al₂O₃ and | | clean liquid | 6.1%, 5.7%, | |
| | Fe₂O₃ | | | 5.1% | |
| 140 | 3 mg | 400° C. | less Black | 34.9%, 16.8%, | 79.2% |
| | Al₂O₃ and | | powder, more | 11.9%, 8.0%, | |
| | Fe₂O₃ | | clean liquid | 7.7% | |

### Example 15

### Experiments 141-195

Wheat straw and corn stover are dried at 50° C. and cut into fragments less than 2.5 cm in length. Aliquots of 0.3 g of these fragments are mixed with 0.1 mole/L, 0.2 mole/L or 0.5 mole/L hydrochloric acid (HCl), sulfuric acid (H₂SO₄), phosphoric acid (H₃PO₄), nitric acid (HNO₃), or trifluoroacetic acid (F₃CCO₂H) and heated at 90° C. for a period of 5, 6 or 16 hours in a glass tube with cape. The resulting reaction mixture is cooled to room temperature and filtered. Filtrations are further used for DNS reducing sugar and total soluble carbohydrate analysis. It shows that at 0.2 mole/L concentration for 5 hours, hydrochloric acid (HCl), nitric acid (HNO₃) or trifluoroacetic acid (F₃CCO₂H) can hydrolyze corn stover to achieve a total reducing sugar yield above 25%. For achieving total reducing sugar yield of above 23% from wheat straw hydrolysis, it requires the incubation with 0.5 mole/L hydrochloric acid (HCl), sulfuric acid (H₂SO₄), nitric acid (HNO₃) or trifluoroacetic acid (F₃CCO₂H) for 5 hours.

In Table 13, the type of feedstock and acid, the initial acid concentration, the length of incubation period, the total reduced sugar yield and the total soluble carbohydrate yield are shown for Experiments 141 to 195.

TABLE 13
Experiments 141 to 195

| | | | | | | Total soluble |
|---|---|---|---|---|---|---|
| | | | Concentration | Incubation period | Total reduced | carbohydrate yield |
| Experiments | Feedstock | Acid | (mole/L) | (hour) | sugar yield (%) | (%) |
| 141 | Wheat straw | HCl | 0.5 | 5 | 30% | 28% |
| 142 | Wheat straw | H₂SO₄ | 0.5 | 5 | 27% | 23% |
| 143 | Wheat straw | HNO₃ | 0.5 | 5 | 24% | 25% |
| 144 | Wheat straw | F₃CCO₂H | 0.5 | 5 | 23% | 21% |
| 145 | Corn Stover | HCl | 0.2 | 5 | 27% | 28% |
| 146 | Corn Stover | H₂SO₄ | 0.2 | 5 | 15% | 21% |
| 147 | Corn Stover | HNO₃ | 0.2 | 5 | 25% | 30% |
| 148 | Corn Stover | F₃CCO₂H | 0.2 | 5 | 25% | 28% |
| 149 | Corn Stover | HCl | 0.5 | 5 | 33% | 30% |
| 150 | Corn Stover | H₂SO₄ | 0.5 | 5 | 32% | 30% |
| 151 | Corn Stover | HNO₃ | 0.5 | 5 | 33% | 32% |
| 152 | Corn Stover | F₃CCO₂H | 0.5 | 5 | 33% | 29% |
| 153 | Wheat straw | HCl | 0.2 | 6 | 19% | 24% |
| 154 | Wheat straw | HNO₃ | 0.2 | 6 | 11% | 16% |
| 155 | Wheat straw | F₃CCO₂H | 0.2 | 6 | 20% | 23% |
| 156 | Wheat straw | HCl | 0.5 | 6 | 29% | 32% |
| 157 | Wheat straw | H₂SO₄ | 0.5 | 6 | 28% | 30% |
| 158 | Wheat straw | HNO₃ | 0.5 | 6 | 24% | 27% |
| 159 | Wheat straw | F₃CCO₂H | 0.5 | 6 | 23% | 25% |
| 160 | Corn Stover | HCl | 0.1 | 6 | 16% | 22% |
| 161 | Corn Stover | HNO₃ | 0.1 | 6 | 11% | 18% |
| 162 | Corn Stover | F₃CCO₂H | 0.1 | 6 | 17% | 25% |
| 163 | Corn Stover | HCl | 0.2 | 6 | 29% | 36% |
| 164 | Corn Stover | H₂SO₄ | 0.2 | 6 | 19% | 25% |
| 165 | Corn Stover | HNO₃ | 0.2 | 6 | 25% | 34% |
| 166 | Corn Stover | F₃CCO₂H | 0.2 | 6 | 28% | 32% |
| 167 | Corn Stover | HCl | 0.5 | 6 | 31% | 34% |
| 168 | Corn Stover | H₂SO₄ | 0.5 | 6 | 32% | 32% |
| 169 | Corn Stover | H₃PO₄ | 0.5 | 6 | 12% | 18% |
| 170 | Corn Stover | HNO₃ | 0.5 | 6 | 35% | 38% |
| 171 | Corn Stover | F₃CCO₂H | 0.5 | 6 | 31% | 34% |
| 172 | Wheat straw | HCl | 0.1 | 16 | 19% | 16% |
| 173 | Wheat straw | F₃CCO₂H | 0.1 | 16 | 17% | 17% |
| 174 | Wheat straw | HCl | 0.2 | 16 | 28% | 24% |
| 175 | Wheat straw | H₂SO₄ | 0.2 | 16 | 17% | 16% |
| 176 | Wheat straw | HNO₃ | 0.2 | 16 | 17% | 18% |
| 177 | Wheat straw | F₃CCO₂H | 0.2 | 16 | 23% | 23% |
| 178 | Wheat straw | HCl | 0.5 | 16 | 31% | 30% |
| 179 | Wheat straw | H₂SO₄ | 0.5 | 16 | 29% | 32% |
| 180 | Wheat straw | H₃PO₄ | 0.5 | 16 | 14% | 14% |
| 181 | Wheat straw | HNO₃ | 0.5 | 16 | 29% | 25% |
| 182 | Wheat straw | F₃CCO₂H | 0.5 | 16 | 25% | 23% |
| 183 | Corn Stover | HCl | 0.1 | 16 | 25% | 22% |
| 184 | Corn Stover | H₂SO₄ | 0.1 | 16 | 21% | 23% |
| 185 | Corn Stover | HNO₃ | 0.1 | 16 | 17% | 17% |
| 186 | Corn Stover | F₃CCO₂H | 0.1 | 16 | 24% | 23% |
| 187 | Corn Stover | HCl | 0.2 | 16 | 32% | 32% |
| 188 | Corn Stover | H₂SO₄ | 0.2 | 16 | 30% | 30% |
| 189 | Corn Stover | HNO₃ | 0.2 | 16 | 31% | 31% |
| 190 | Corn Stover | F₃CCO₂H | 0.2 | 16 | 29% | 30% |
| 191 | Corn Stover | HCl | 0.5 | 16 | 35% | 36% |
| 192 | Corn Stover | H₂SO₄ | 0.5 | 16 | 33% | 33% |
| 193 | Corn Stover | H₃PO₄ | 0.5 | 16 | 22% | 23% |
| 194 | Corn Stover | HNO₃ | 0.5 | 16 | 37% | 37% |
| 195 | Corn Stover | F₃CCO₂H | 0.5 | 16 | 32% | 31% |

### Example 16

### Experiments 196-223

Wheat straw and corn stover are dried at 50° C. and cut into fragments less than 2.5 cm in length. Aliquots of 0.3 g of these fragments are mixed with 0.1 mole/L, 0.2 mole/L, or 0.5 mole/L hydrochloric acid (HCl), sulfuric acid (H₂SO₄), phosphoric acid (H₃PO₄), nitric acid (HNO₃), or trifluoroacetic acid (F₃CCO₂H) and heated at 90° C. for a period of 5, 6, or 16 hours in a glass tube with cape. The resulting reaction mixture is then cooled to room temperature and filtered. The wet solid fragments are heated at 160° C. for 2 hours and 4 hours in a hydrothermal reaction vessel. After the cooling, the solid residues are put in mortar to test the degree of easiness for grinding into fine fragments.

If it takes more than 10 minutes to grind the biomass fragments in a mortar into fine particles, the degree of easiness of grinding is denoted as “+”. If it takes less than 10 seconds to grind such fragments into fine particles, the degree of easiness of grinding is denoted “+++++”.

In Table 14, the type of feedstock and acid, the initial acid concentration, and the degree of easiness of grinding are shown for Experiments 196 to 215.

After incubating with 0.1 mole/L trifluoroacetic acid at 160° C. for 2 hours, wheat straw and corn stover are very easy to grind. After incubating with 0.2 mole/L hydrochloric acid at 160° C. for 2 hours, corn stover is very easy to grind, while wheat straw needs to be conditioned at 160° C. for 4 hours to be very easy to grind. After incubating with 0.1 and 0.2 mole/L phosphoric acid at 160° C. for 4 hours, wheat straw and corn stover, respectively, are very easy to grind.

TABLE 14
Experiments 196 to 215

| | | | Concentration | Time | Easiness of |
|---|---|---|---|---|---|
| Experiments | Feedstock | Acid | (mole/L) | (hour) | grinding |
| 196 | Wheat straw | HCl | 0.1 | 2 | +++ |
| 197 | Wheat straw | H₂SO₄ | 0.1 | 2 | ++ |
| 198 | Wheat straw | H₃PO₄ | 0.1 | 2 | ++ |
| 199 | Wheat straw | HNO₃ | 0.1 | 2 | +++ |
| 200 | Wheat straw | F₃CCO₂H | 0.1 | 2 | ++++ |
| 201 | Wheat straw | HCl | 0.2 | 2 | +++ |
| 202 | Wheat straw | H₂SO₄ | 0.2 | 2 | ++ |
| 203 | Wheat straw | H₃PO₄ | 0.2 | 2 | ++ |
| 204 | Wheat straw | HNO₃ | 0.2 | 2 | +++ |
| 205 | Wheat straw | F₃CCO₂H | 0.2 | 2 | ++++ |
| 206 | Corn Stover | HCl | 0.1 | 2 | +++ |
| 207 | Corn Stover | H₂SO₄ | 0.1 | 2 | +++ |
| 208 | Corn Stover | H₃PO₄ | 0.1 | 2 | ++ |
| 209 | Corn Stover | HNO₃ | 0.1 | 2 | +++ |
| 210 | Corn Stover | F₃CCO₂H | 0.1 | 2 | +++ |
| 211 | Corn Stover | HCl | 0.2 | 2 | ++++ |
| 212 | Corn Stover | H₂SO₄ | 0.2 | 2 | +++ |
| 213 | Corn Stover | H₃PO₄ | 0.2 | 2 | +++ |
| 214 | Corn Stover | HNO₃ | 0.2 | 2 | ++++ |
| 215 | Corn Stover | F₃CCO₂H | 0.2 | 2 | ++++ |
| 216 | Wheat straw | HCl | 0.1 | 4 | ++++ |
| 217 | Wheat straw | HCl | 0.2 | 4 | ++++ |
| 218 | Wheat straw | H₃PO₄ | 0.1 | 4 | ++++ |
| 219 | Wheat straw | H₃PO₄ | 0.2 | 4 | ++++ |
| 220 | Corn Stover | HCl | 0.1 | 4 | ++++ |
| 221 | Corn Stover | HCl | 0.2 | 4 | ++++ |
| 222 | Corn Stover | H₃PO₄ | 0.2 | 4 | ++++ |
| 223 | Corn Stover | H₃PO₄ | 0.2 | 4 | ++++ |

### Example 17

### Experiments 224-230

Wheat straw is dried at 50° C. and cut into fragments less than 2.5 cm in length. Aliquots of 20 g of these fragments are taken and mixed with 400 ml 1.0% trifluoroacetic acid (TFA) and then heated at 90° C. for 4 hours in a hydrothermal reaction vessel. The reaction mixture is cooled to room temperature and then filtered. The filtrate is analyzed for reducing sugar and total carbohydrate. The resulting amount of total carbohydrate is 4.01 g, and the average polymerization degree of xylose polymer and/or xylose oligomer is 2.4.

7 aliquots of 5 ml filtrate are taken and mixed well with ethanol to final concentration of 30%, 50%, 60%, 70%, 80%, 85%, and 90% (v/v), and then stand still over night. The mixture is then centrifuged and the resulting precipitate is collected and washed twice with 95% ethanol, and dried. The dry precipitate is analyzed for reducing sugar and total carbohydrate and average polymerization.

In Table 16, the initial concentration of ethanol, the reducing sugar and total carbohydrate, and average polymerization are listed.

TABLE 16
Experiment 224-230

| | Ethanol | | Total | |
|---|---|---|---|---|
| | concentration | Reducing | carbohydrate | Average |
| Experiments | (% v/v) | sugar (mg) | (mg) | polymerization |
| 224 | 30 | 11.2 | 14.4 | 1.3 |
| 225 | 50 | 5.4 | 11.4 | 2.1 |
| 226 | 60 | 6.6 | 17.6 | 2.7 |
| 227 | 70 | 16 | 36 | 2.3 |
| 228 | 80 | 9.8 | 50.8 | 5.2 |
| 229 | 85 | 9.7 | 54.4 | 5.6 |
| 230 | 90 | 13.3 | 57.9 | 4.3 |

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure vary substantially without departing from the spirit of the invention, and exclusive use of all modifications that come within the scope of the appended claims is reserved. It is intended that the present invention be limited only to the extent required by the appended claims and the applicable rules of law.

All literature and similar material cited in this application, including, patents, patent applications, articles, books, treatises, dissertations, web pages, figures and/or appendices, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

1. A process for converting biomass into soluble and insoluble fractions, said process comprising:
(i) incubating the biomass with an aqueous acidic liquor;
(ii) separating the acidic liquor from the resulting solid residue of the biomass, wherein the acidic liquor contains the soluble fraction and the solid residue contains the insoluble fraction; and
(iii) collecting the products of the biomass conversion.

2. The process of claim 1, wherein said incubation with the acidic liquor produces a soluble product, or a combination of products, separated from the biomass and dissolved in the acidic liquor.

3. The process of claim 1, wherein the acidic liquor is recovered after step (ii) for reuse.

4. A process for converting biomass into soluble and insoluble products, said process comprising:
(i) incubating the biomass with an aqueous acidic liquor so that a soluble product, or a combination of soluble products, are separated from the biomass and dissolved in the acidic liquor;
(ii) separating the acidic liquor containing the soluble product(s) from the resulting solid residue of the biomass;
(iii) recovering the soluble products from the acidic liquor;
(iv) recovering the acidic liquor from the separated liquid and the solid residue for reuse;
(v) grinding the insoluble solid residue recovered from step (ii) and further hydrolyzing the insoluble solid residue with cellullase enzyme; and
(vi) recovering the resulting soluble products and the insoluble lignin products from steps (ii)-(iii) and the resulting solid residue.

5. The process of claim 1, wherein said biomass comprises a lignocellulosic biomass feedstock.

6. A process for converting lignocellulosic biomass feedstock into lignocellulosic products, the process comprising:
(i) incubating the lignocellulosic biomass feedstock with an acidic liquor comprising an acid or a combination of acids so that a soluble product, or a combination of products, are separated from the biomass and dissolved in the acid(s);
(ii) separating the resulting liquid from the resulting solid residue, wherein the liquid contains the soluble lignocellulosic products and the solid residue contains the insoluble lignocellulosic fraction;
(iii) recovering the soluble lignocellulosic products from the separated liquid;
(iv) recovering the acidic liquor from the separated liquid for reuse;
(v) grinding the insoluble lignocellulosic solid fraction recovered in step (iii) and further hydrolyzing the insoluble lignocellulosic solid residue with cellullase enzyme; and
(vi) recovering the resulting soluble products and the insoluble lignin products as in steps (ii)-(iii) and the resulting solid residue.

7. The process of claim 1, wherein said biomass is selected from the group consisting of woody plants, gramineous plants, and herbage plants, or a combination thereof.

8. The process of claim 1, wherein said biomass is converted to products selected from the group comprising of cellulose, hemicellulose, xylose polymer, xylose oligomer, xylose monomer, glucose, lignin, and other lignocellulosic products, or a combination thereof.

9. The process of claim 8, wherein said converted products are further converted to bioenergy, biochemicals, or other bulk materials, or a combination thereof.

10. The process of claim 1, wherein said acidic liquor in step (i) comprises a low-boiling point organic acid or an inorganic (mineral) acid, or a combination thereof.

11. The process of claim 10, wherein said organic acid in step (i) is selected from the group consisting of formic acid, acetic acid, 2-hydroxypropionic acid, propionic acid, acrylic acid, propylene-2-carboxylic acid, n-pentanoic acid, lactic acid, trifluoromethane sulfonic acid, methyl acrylic acid and trifluoroacetic acid (TFA), or a combination thereof.

12. (canceled)

13. The process of claim 10, wherein said inorganic acid in step (i) is selected from the group consisting of hydrochloric acid, sulfuric acid, phosphoric acid, and nitric acid, or a combination thereof.

14. A composition comprising fractions produced by the process of claim 1.

15. (canceled)

16. The process of claim 1, wherein in step (i) said biomass is incubated with an aqueous acidic liquor comprising 0.1%-100% dilute acid.

17. The process of claim 16, wherein the aqueous acidic liquor comprises 0.1%-5.0% dilute acid.

18. The process of claim 1, wherein in step (i) said biomass is incubated at about 50° C.-160° C.

19. The process of claim 1, wherein the soluble products converted from said biomass in step (vi) is glucose with a purity of at least 90%.

20. The process of claim 1, wherein the insoluble products converted from said biomass in step (vi) is lignin with a purity of at least 90%;

21. The process of claim 6, wherein said lignocellulosic biomass feedstock comprises fragmentated feedstock.

22. The process of claim 6, wherein said lignocellulosic biomass is capable of passing through about 8-64 mesh filters.

23. The process of claim 6, wherein the weight:volume ratio of said lignocellulosic biomass feedstock vs. acid is 1:2-1:20.

24. The process of claim 6, wherein in step (i) said lignocellulosic biomass feedstock is incubated for 1-16 hours.

25-26. (canceled)

27. The process of claim 6, wherein in step (i) said lignocellulosic biomass feedstock is incubated with 0.1%-5% dilute acid at 50° C.-90° C. and in step (v) the insoluble lignocellulosic solid fraction is conditioned before grinding at temperature 120° C.-160° C.

28. The process of claim 6, wherein the separated liquid in step (iii) contains soluble xylose polymer, xylose oligomer, or xylose monomer, or a combination thereof, in the concentration of at least 90%.

29. The process of claim 27, wherein the insoluble lignoscellulosic solid fraction conditioned in step (v) is heated for 3-6 hours.

30. The process of claim 6, wherein in step (v) said hydrolysis comprises an incubation with cellulase at 10° C. to 90° C.

31. The process of claim 6, wherein in step (v) cellulase is used for hydrolysis to produce soluble products comprising glucose, wherein the concentration of produced glucose is at least 90%.

32. The process of claim 6, wherein in step (v) cellulase is used for hydrolysis and the resulting solid residue comprises lignin, wherein the concentration of lignin in said solid residue is at least 90%.

33. The process of claim 6, wherein the recovered lignin products are further converted into lignin-related products.

34. The process of claim 33, wherein the recovered lignin products are heated with or without catalyst to 300° C.-500° C.

35. The process of claim 33, wherein the recovered lignin products are heated to 450° C. under vacuum for 12 hours to produce a resulting liquid comprising at least 5 compounds with a total concentration of at least 75%.

36. The process of claim 33, wherein the recovered lignin products are mixed with Al₂O₃ and Fe₂O₃ as catalyst and heated to 400° C. under vacuum for 12 hours to produce a resulting liquid comprising at least 5 compounds with a total concentration of at least 79%.

37. A process for converting lignocellulosic biomass feedstock into bioenergy, biochemicals, or other bulk materials, the process comprising:
(i) preparing xylose products using the processes of any one of the above claims;
(ii) culturing at least one species of microbes in a broth containing the prepared xylose products;
(iii) fermenting the composition of step (ii); and
(iv) collecting bioenergy, biochemicals, or other bulk materials, or a combination thereof, from the fermentation broth.

38. The process of claim 37, wherein said microbes comprise Pichia pastoris GS115.

39. The process of claim 37, wherein said fermentation of the microbe culture is used for said conversion.

40. A process for converting lignocellulosic biomass feedstock into xylose oligomer, the process comprising:
(i) preparing soluble lignocellulosic products using the process of claim 6; and
(ii) separating xylose oligomers from the soluble lignocellulosic products in step (i).

41. The process of claim 40, wherein said separation is through ethanol precipitation.

42. The process of claim 41, wherein the concentration of ethanol is 30%-90%.

43. The process of claim 40, wherein the average degree of polymerization of xylose oligomers is 1.3-5.6.

44. A lignocellulosic composition of products produced by the process of claim 37.

45. A lignocellulosic composition comprising at least 90% of xylose polymers, xylose oligomers, xylose monomers, or a combination thereof, produced by the process of claim 6.

46. A lignocellulosic composition comprising at least 90% of glucose, produced by the process of claim 6.

47. A lignocellulosic composition comprising at least 90% of lignin, produced by the process of claim 6.

48. A lignocellulosic feedstock processing system comprising a set of devices capable of carrying out the process of claim 1.

49. The lignocellulosic feedstock processing system of claim 48, wherein said system further comprises a feedstock handling device and a preconditioner capable of receiving said feedstock from said handling device, wherein said preconditioner is in communication with said set of devices of claim 48.
